(19) United States
(12) Patent Application Publication
(10) Pub. No.: US 2023/0310467 A1
(43) Pub. Date: Oct. 5, 2023

(54) PHARMACEUTICAL COMBINATION THERAPY AND PREVENTION WITH APROTININ + REMDESIVIR OF SARS-CoV-2 AND/OR DISEASE ASSOCIATED WITH THIS INFECTION, INCLUDI COVID-19

(71) Applicants: Alexandre Vasilievich Ivachtchenko, Hallandale, FL (US); Alena Alexandrovna Ivachtchenko, Hallandale, FL (US); Nikolay Filippovich Savchuk, Rancho Santa Fe, CA (US); ASAVI LLC, Hallandale, FL (US)

(72) Inventors: Alexandre Vasilievich Ivachtchenko, Hallandale, FL (US); Andrey Alexandrovich Ivashchenko, Moscow (RU); Alena Alexandrovna Ivachtchenko, Hallandale, FL (US); Ilya Andreevich Ivashchenko, Moscow region (RU); Nikolay Filippovich Savchuk, Rancho Santa Fe, CA (US)

(73) Assignees: ALEXANDRE VASILIEVICH IVACHTCHENKO, Hallandale, FL (US); ALENA ALEXANDROVNA IVACHTCHENKO, Hallandale, FL (US); NIKOLAY FILIPPOVICH SAVCHUK, Rancho Santa Fe, CA (US); ASAVI LLC, Hallandale, FL (US)

(21) Appl. No.: 17/709,609

(22) Filed: Mar. 31, 2022

(51) Int. Cl. A61K 31/675; A61K 31/519; A61K 38/57; A61P 31/14

(52) U.S. Cl. CPC A61K 31/675; A61K 31/519; A61K 38/57; A61P 31/14

(57) ABSTRACT

The application relates to pharmaceutical combinations, methods, and therapies for the treatment and prevention of SARS-CoV-2 infection and related diseases. The combinations contain aprotinin, baricitinib, and/or remdesivir. The application also includes related pharmaceutical kits. Further details are described in the specification.

### FIELD OF INVENTION

The present invention is directed to a new pharmaceutical combination therapy with aprotinin+remdesivir, a pharmaceutical kit and a pharmaceutical composition intended for the prevention and treatment of severe acute respiratory syndrome (SARS), as well as diseases caused by SARS, including coronavirus diseases.

### BACKGROUND OF THE INVENTION

The RNA viruses of SARS viruses have a tropism for the epithelium of the mucous membranes of the respiratory system. They are characterized by catarrhal damage to the mucous membranes of the larynx, trachea, bronchi with involvement of the lungs in the process. The infections are transmitted mainly by aerosol transmission.

Severe Acute Respiratory Syndrome Coronavirus 2 (SARS-CoV-2) is RNA virus that causes a 2019 Coronavirus Disease (COVID-19). SARS-CoV-2 is responsible for the ongoing COVID-19 pandemic. SARS-CoV-2 is a virus that belongs to a type of coronavirus associated with SARS-CoV (Coronaviridae Study Group of the International Committee on Taxonomy of Viruses (April 2020). “The species Severe acute respiratory syndrome-related coronavirus: classifying 2019-nCoV and naming it SARS-CoV-2”. Nat. Microbiol. 2020, 5(4), 536-544. This virus was first identified in December 2019 in Wuhan city, Hubei province, China. On Mar. 11, 2020, WHO declared the outbreak a public health emergency of international concern. SARS-CoV-2 is the successor to the SARS-CoV-1 virus that caused the SARS outbreak in 2002-2004 (“New coronavirus stable for hours on surfaces”. National Institutes of Health (NIH). NIH.gov. 17 Mar. 2020. Archived from the original on 23 Mar. 2020. Retrieved 4 May 2020). SARS-CoV-2 has undergone many changes in two years, and each new mutation has been more perfect than the previous one. First discovered in India in December 2020, the Delta mutation is spreading across continents at an alarming rate. Delta penetrates lung cells more easily than the original virus (the virus that circulated in the early stages of the pandemic). In addition, the Delta strain is more effective in combining infected lung cells with uninfected ones. This could contribute to the more severe course of COVID-19. It is currently the predominant variant of SARS-CoV-2 worldwide. Delta is believed to be more than twice as infectious as previous SARS-CoV-2 variants (K. Katella. 5 Things to Know About the Delta Variant. Yale Medicine NOV. 19, 2021.

The new variant of coronavirus Omicron was detected in laboratories in Botswana and South Africa on 22 Nov. 2021. The variant has an unusually large number of mutations, several of which are novel and a significant number of which affect the spike protein targeted by most COVID-19 vaccines at the time of discovering the Omicron variant. This level of variation has led to concerns regarding its transmissibility, immune system evasion, and vaccine resistance. Omicron spreads faster than any previously known variant.

Dec. 17, 2021 77 countries have now reported cases of Omicron, and “the reality is that Omicron is probably in most countries, even if it hasn't been detected yet (L. Smith-Spark, What can the world learn from countries where Omicron is surging? CNN Fri Dec. 17, 2021.

As of Feb. 11, 2022, 404,910,528 confirmed cases of people infected with coronavirus were registered in the world, of which 5,783,776 died.

Vaccination remains one of the main public health interventions to combat SARS -CoV-2. However, vaccine development times of at least six months limit their applicability during outbreaks of new strains of SARS-CoV-2, like the Omicron strain. Therefore, the new development of new highly effective anticoronavirus drugs is an urgent task.

The first of the closest to this invention is a pharmaceutical combination therapy with oral baricitinib (BCN, Olumiant®) and intravenous remdesivir (RDV, VEKLURY®) of suspected or laboratory-confirmed COVID-19 in hospitalized people requiring supplemental oxygen, invasive mechanical ventilation, or extracorporeal membrane oxygenation (Kalil A. C. et al. Baricitinib plus Remdesivir for Hospitalized Adults with Covid-19. N. Engl. J. Med. 2021, 384, 795-807.

In November 2020, FDA issued an emergency use authorization (EUA) for the combination of the drugs BCN and RDV, for the treatment of moderate to severe COVID-19 patients two years of age or older (FDA. Coronavirus (COVID-19) Update: FDA Authorizes Drug Combination for Treatment of COVID-19. Press release 19 Nov. 2020.

It should be noted that the patient population treated with BCN in combination with RDV improved only modestly median time to recovery from 8 to 7 days in comparison to remdesivir, a 12.5% improvement (Kalil A. C. et al. Baricitinib plus Remdesivir for Hospitalized Adults with Covid-19. N. Engl. J. Med. 2021, 384, 795-807.

The second of the closest to this invention is a method of treating of moderate COVID-19 patients by the intravenous aprotinin (APR) and oral avifavir (favipiravir, FVP) combination therapy. This therapy is more effective because primary and secondary efficacy endpoints of therapy by the APR+FVP combination significantly better than efficacy endpoints of therapy by the individual components (Table 1). (Ivashchenko A. A. et al., Effect of Aprotinin and Avifavir® Combination Therapy for Moderate COVID-19 Patients. Viruses 2021, 13, 1253.

TABLE 1
Primary and secondary efficacy endpoints of therapy
by the intravenous aprotinin (APR), oral avifavir
(favipiravir, FVP) and their combination.

| | | | APR + |
|---|---|---|---|
| Primary and Secondary Efficacy | APR + | FVP + | FVP + |
| Endpoints | SOC | SOC) | SOC |
| Median time to elimination of SARS- | 7.5 (6- | 4.5 (4- | 3.5 (3- |
| CoV-2 confirmed by RT-PCR, days | 9) | 9) | 4) |
| (IQR) | | | |
| Median time to normalization of CRP | 6.0 (6- | 14.0 (5.5- | 3.5 (3- |
| concentration (≤10 mg/L) in patient's | 6) | 14) | 5) |
| blood, days (IQR) | | | |
| Median time to normalization of D- | 4.5 (3- | NA | 5.0 (4- |
| dimer concentration (<253 ng/mL) in | 6) | | 5) |
| patient's blood, days (IQR) | | | |
| Median time to normalization of body | 3.0 (2- | 2.0 (1- | 1.0 (1- |
| temperature (<37° C.), days (IQR) | 3) | 3) | 3) |
| Median time to improvement in clinical | 11.0 (6- | 14.0 (11.5- | 5.0 (5- |
| status by 2 points on the WHO-OSCI, | 11) | 16) | 5) |
| days (IQR) | | | |

### TERMS USED IN THE DESCRIPTION OF THIS INVENTION

The term “drug” (also called medicine, medicament, pharmaceutical composition, or medicinal drug) refers to a drug used to diagnose, cure, treat, or prevent disease and means a substance (or a mixture of substances in the form of a pharmaceutical composition).

The term “oral drug” refers to solutions, powders, tablets, capsules, and pills that are taken by mouth and swallowed.

The term “parenteral drug” refers to drugs that are injected into the body bypassing the gastrointestinal tract. Parenteral drugs are solutions for injection, inhalation, sprays, including for nasal or drip application, and other finished dosage forms, in this case intended for the treatment and prevention of viral infections and diseases caused by them.

The term “parenteral therapies” are administration of drugs is primarily injections (intravenously, into the muscles, under the skin), inhalations and nasally (spray, drops).

The term “pharmaceutical composition” as used herein means a composition comprising at least two active ingredients (substances), namely APR an RDV, and at least one excipient.

The term “parenteral pharmaceutical composition (PPC)” is intended for parenteral administration of drugs into the body of a patient. These are primarily intravenous, inhalation and nasal routes of drug administration.

The term “excipient” as used herein refers to a compound that is used to prepare a pharmaceutical composition and is generally safe, non-toxic, and neither biologically nor otherwise undesirable, and includes excipients that are acceptable to humans and animals. This invention uses primarily excipients selected from the series: water, sodium chloride, L-lysine monohydrate, sodium hydroxide, hydrochloric acid, benzyl alcohol, ethanol, glycerin, dimethyl sulfoxide, peppermint oil, 1,1,1,2-tetrafluoroethane, and some others.

The term “pharmaceutical kit” as used herein means a kit including at least two drugs: Bexovid® or NMV saline solution, or its lyophilizate and the parenteral drug Trasylol®, Gordox®, Aprotex®, Antagosan®, Contrycal®, Traskolan®, or others parenteral drugs including aprotinin, or aqueous or saline solution containing aprotinin.

The term “pharmaceutical combination therapy” is therapy that uses more than one pharmaceutical medication (drug). “Pharmaceutical combination therapy” may be achieved by prescribing/administering separate drugs, or dosage forms that contain more than one active ingredient (such as fixed-dose combinations).

The term “combination therapy” is therapy that uses more than one medication or modality. Typically, the term refers to using multiple therapies to treat a single disease, and often all the therapies are pharmaceutical. “Pharmaceutical” combination therapy may be achieved by prescribing/administering separate drugs, or, where available, dosage forms that contain more than one active ingredient (such as fixed-dose combinations).

The term “therapeutically effective amount” or “dose” as used herein means the amount of medicine needed to reduce the symptoms of a disease in a patient. The dose of medicine will be tailored to the individual requirements in each case. This dose can vary widely depending on numerous factors, such as the severity of the patient's illness, the age and general health of the patient, other drugs with which the patient is being treated, the method and form of administration of medicine, and the experience of the attending physician. Typically, treatment is started with a large initial “loading dose” to rapidly reduce or eliminate the virus and followed by tapering the dose to a level sufficient to prevent an outbreak of infection.

The term “patient” means a mammal including but not limited to humans, cattle, pigs, sheep, chickens, turkeys, buffaloes, llamas, ostriches, dogs, cats, hamsters, and mice, preferably the patient is a human.

The term “active ingredient (substance)” as used herein means aprotinin and an inhibitor of RNA viruses used in a pharmaceutical composition or drug.

### SUMMARY OF THE INVENTION

The first aspect of the invention relates to the pharmaceutical combination therapy and prevention of SARS-CoV-2 and/or disease associated with this infection, including COVID-19, using aprotinin (APR), baricitinib (BCN) and/or remdesivir (RDV).

Another aspect of the invention is a pharmaceutical kit for a pharmaceutical combination treatment and prevention of SARS-CoV-2 and/or disease associated with this infection, including viral and bacterial pneumonia and COVID-19, consisting of the parenteral drug Trasylol®, Gordox®, Aprotex®, Antagosan®, Contrycal®, Traskolan®, other parenteral drug that include aprotinin, an aqueous or saline solution containing aprotinin and VEKLURY®, other oral drug that include remdesivir, aqueous or saline solution of remdesivir, or their lyophilizate and, optionally, Olumiant® or aqueous or saline solution containing baricitinib, or its lyophilizate for parenteral administration, which includes baricitinib.

Another aspect of the invention is a pharmaceutical composition in the form of an aqueous solution (APC) or lyophilizate (PCL) for a pharmaceutical combination therapy and prevention of SARS-CoV-2 and/or disease associated with this infection, including COVID-19, including APR, BCN and/or RDV and excipients.

The excipients are selected from the series: water, sodium chloride, L-lysine monohydrate, 2-hydroxy-beta-cyclodextrin, betadex sulfobutyl ether sodium, sodium hydroxide, hydrochloric acid, benzyl alcohol, ethanol, glycerin, dimethyl sulfoxide, peppermint oil, 1,1,1,2-tetrafluoroethane, and others.

APR is an inhibitor of the natural proteases with a long history of clinical use since the 1960s and a good safety profile. APR, under the trade names Trasylol®, Gordox®, Aprotex®, Antagosan®, Contrycal®, Traskolan®, and others, is used as an intravenous medication given by injection to reduce bleeding during complex surgeries such as heart and liver surgery, as an antiviral drug for the treatment and prevention of viral respiratory diseases (U.S. Pat. No. 5,723,439).

Parenteral drugs Trasylol®, Gordox®, Aprotex®, Antagosan®, Contrycal®, Traskolan®, and others are aqueous solutions of APR with an APR activity of 5000-10000 KIU/ml containing excipients selected from the series: sodium chloride, sodium hydroxide, 1M hydrochloric acid solution, benzyl alcohol, and others. These parenteral drugs are used to prevent intraoperative blood loss and reduce the volume of blood transfusion in liver and heart transplants, coronary artery bypass grafting using a heart-lung machine in adult patients who are at increased risk of bleeding or need blood transfusion. These drugs are also recommended as a preventive treatment for patients who are likely to be at an increased risk of bleeding or need transfusion.

APR inhibits the entry of SARS-CoV-2 into cells ((a) M. Hoffmann et al. SARS-CoV-2 Cell Entry Depends on ACE2 and TMPRSS2 and Is Blocked by a Clinically Proven Protease Inhibitor. Cell 2020, 181 (2), 271-280. e8; (b) D. Bojkova et al. Aprotinin Inhibitors SARS-CoV-2 Replication. Cells 2020, 9 (11), 2377., and can be used for the prevention and treatment of SARS-20 CoV-2/COVID-19 (RU 2738885).

RDV (VEKLURY®, GS-5734) was originally developed over a decade ago for the treatment of hepatitis C and RSV. RDV has been reported to be active against the coronavirus MERS-CoV, SARS-CoV, CoV-OC43, CoV-229E, and PDCoV.35 (A. Frediansyah, et al. Remdesivir and its antiviral activity against COVID-19: A systematic review. Clin. Epidemiol. Glob. Health 2021, 9, 123-127. RDV for IV infusion is available in two dosage forms: in the form of a concentrate (5 mg/mL of remdesivir) for preparing a solution for IV infusion and in the form of a lyophilizate (100 mg/vial remdesivir) for preparing a concentrate for an IV infusion solution Remdesivir Gilead, INN-remdesivir (europa.eu); The inactive ingredients of VEKLURY (RDV) for injection are sulfobutylether-β-cyclodextrin (SBECD) sodium salt, Water for Injection, USP, and may include hydrochloric acid and/or sodium hydroxide for pH adjustment. VEKLURY for injection, 100 mg, contains 3 g SBECD, and VEKLURY injection, 100 mg/20 mL (5 mg/mL), contains 6 g SBECD.

BCN, sold under the brand name Olumiant among others, is a drug for the treatment of rheumatoid arthritis (RA) in adults whose disease was not well controlled by tumor necrosis factor (TNF) inhibitors (FDA, 2018. Drug Trials Snapshots: Olumiant. It acts as an inhibitor of janus kinase (JAK), blocking the subtypes JAK1 and JAK2 (EMA, 2016. Summary of opinion for Olumiant

The drug is approved for medical use in the European Union and in the United States. On 31 May 2018, the FDA approved barictinib for the treatment of adults with moderately to severely active rheumatoid arthritis who have had an inadequate response to one or more TNF antagonist therapies.

Serious side effects have occurred in patients treated with OLUMIANT: cardiovascular events, thrombosis, malignancy, serious bacterial, fungal, viral infections, including tuberculosis, leading to hospitalization or death.

In April 2020, Lilly announced they are investigating the use of baricitinib for treating people with COVID-19. The drug's anti-inflammatory activity is expected to act on the inflammatory cascade associated with COVID-19 (Eli Lilly to study baricitinib for Covid-19 treatment”.

In November 2020, published research showed barcitinib was beneficial in treating people with COVID-19. According to the paper “mechanistic actions of a Janus kinase-1/2 inhibitor targeting viral entry, replication and the cytokine storm, and is associated with beneficial outcomes including in severely ill elderly people” (Stebbing J. et al. (January 2021). JAK inhibition reduces SARS-CoV-2 liver infectivity and modulates inflammatory responses to reduce morbidity and mortality”. Science Advances. 2021, 7 (1), eabe 4724.

In a clinical trial of hospitalized people with COVID-19, baricitinib, in combination with remdesivir, was shown to reduce time to recovery within 29 days after initiating treatment compared to participants who received a placebo with remdesivir. The safety and effectiveness of this investigational therapy for use in the treatment of COVID-19 continues to be evaluated (FDA, 2020. Coronavirus (COVID-19) Update: FDA Authorizes Drug Combination for Treatment of COVID-19”.

Another aspect of the invention is directed to anti-SARS-CoV-2 viral disease and/or disease associated with this infection, including COVID-19, parenteral drug, which is APC of this invention.

The parenteral drug and APC according to this invention is intended for the prevention and treatment mainly of SARS-CoV-2 and COVID-19.

Another aspect of the invention is directed to the containing APC of this invention container.

Another aspect of the invention is directed to an ampoule and a bottle containing APC of this invention.

Another aspect of the invention is directed to an inhaler (nebulizer) selected from a number of compressor or ultrasonic or electronic mesh inhaler (nebulizer) containing APC of this invention.

Another aspect of the invention is directed to a pocket or portable inhaler (nebulizer) containing APC of this invention.

Another aspect of the invention is directed to a container, which is a can for nasal spray containing APC of this invention.

Another aspect of the invention is directed to a method for the prevention and/or treatment of SARS-CoV-2 infection and/or disease associated with this infection in a patient by parenteral (intravenous, inhalation, or intranasal) administration to the patient of a saline solution APR and oral BCN and/or RDV and in a therapeutically effective amount once or twice a day (as prescribed by a physician, depending on the patient's condition).

Another aspect of the invention is directed to the use of the parenteral saline solution APR and oral FVP and/or MPV in a therapeutically effective amount once or twice a day (as prescribed by a physician, depending on the patient's condition) for the prevention and/or treatment of SARS-CoV-2 infection and/or disease associated with this infection, including COVID-1.

Another aspect of the invention is directed to the use of the parenteral saline solutions APR, BCN and/or RDV in a therapeutically effective amount once or twice a day (as prescribed by a physician, depending on the patient's condition) for the prevention and/or treatment of SARS-CoV-2 infection and/or disease associated with this infection, including COVID-1. Instead of saline solution of APR can be used Trasylol®, Gordox®, Aprotex®, Antagosan®, Contrycal®, Traskolan®, or others are aqueous solutions of APR.

Another aspect of the invention is directed to a method for the prevention and treatment by administration to the patient in a therapeutically effective amount once or twice a day (as prescribed by a physician, depending on the patient's condition) of the parenteral drug Trasylol®, Gordox®, Aprotex®, Antagosan®, Contrycal®, Traskolan®, other parenteral drug that include aprotinin, or an aqueous or saline solution containing aprotinin, Olumiant® or aqueous or saline solution containing baricitinib, or its lyophilizate, and/or VEKLURY® or aqueous or saline solution containing remdesivir or its lyophilizate, and if necessary, excipients.

Another aspect of the invention is directed to a method for the prevention and treatment by parenteral (intravenous, inhalation, nasal) administration to the patient in a therapeutically effective amount once or twice a day of the aqueous pharmaceutical composition which includes in a therapeutically effective amount aprotinin, baricitinib and/or remdesivir and, if necessary, excipients.

In accordance with this invention, prophylaxis of patients is carried out with a therapeutically effective amount of a nasal spray, which is APC of this invention.

In accordance with this invention, prophylaxis is carried out using nasal spray cans in the nose (nasopharynx) and throat 3-6 times a day in each nostril and throat with a therapeutically effective amount of APC according to the present invention.

In accordance with this invention, the prophylaxis and treatment of patients is carried out by inhalation using ultrasonic nebulizers 3-6 times a day for 5-7 days a therapeutically effective amount of APC according to this invention.

Another aspect of the invention is directed to the use of the APC or the parenteral drug of the present invention for the prevention and/or treatment of SARS-CoV-2 and/or disease associated with this infection by intravenous, inhalation, or intranasal administration to a patient of the APC or the parenteral drug of the present invention in a therapeutically effective amount.

Another aspect of the invention is directed to the use of the APC or the parenteral drug of the present invention in a therapeutically effective amount for the prevention and/or treatment of SARS-CoV-2 and/or disease associated with this infection, including COVID-19.

The use of the new pharmaceutical kit and APC and the new parenteral drug of the present invention in pharmaceutical combination therapy and prevention of SARS-CoV-2 and/or disease associated with this, including COVID-19, was more effective than monotherapy with BCN and/or RDV.

On the one hand, when using APC or the separate saline solutions APR, BCN and/or RDV, additionally significantly simplifies the process of prevention and treatment of patients compared with separate combination therapy by the oral drug+the parenteral drug and allows treatment of patients unable to take oral drugs.

In addition, the use of the new pharmaceutical combination therapy of the present invention is more effective against SARS-CoV-2, since it uses drugs with two different mechanisms of action. APR is a nonspecific inhibitor of the serine proteases-especially trypsin, chymotrypsin, plasmin, kallikrein, and an inhibitor of entry of SARS-CoV-2 into the cell. BCN is an inhibitor of janus kinase (JAK), blocking the subtypes JAK1 and JAK2 and RDV is inhibitors of a replication of SARS-CoV-2 in cells.

In addition, the use of the new pharmaceutical combination therapy of the present invention provides through the use of APR effective inhibition of inflammation and thrombosis in the moderate and severe COVID-19 patients.

Another aspect of the invention is directed to a method of obtaining the APC or the parenteral drug of the present invention by dissolving APR, BCN and/or RDV, and excipients in saline (0.9% aqueous sodium chloride solution).

Another aspect of the invention is directed to a method of obtaining APC or the new parenteral drug of the present invention by dissolving a lyophilizate containing APR, BCN and/or RDV and excipients in saline.

The active ingredients and the PCL of the present invention retain their activity in a convenient lyophilized form for storage.

Another aspect of the invention is directed to the use of APR in the form of a powder a lyophilizate, a concentrate, or a drug selected from the group consisting of Trasylol®, Gordox®, Aprotex®, Antagosan®, Contrycal®, Traskolan®, and others for preparing the APC or the parenteral drug of the present invention.

Another aspect of the invention is directed to the use of BCN and/or RDV in the form of a powder, or a lyophilizate, or a concentrate for preparing the APC or the parenteral drug of the present invention.

Another aspect of the invention is directed to the use of BCN and/or RDV in the form of a powder, or a lyophilizate, or a concentrate and APR as Trasylol®, Gordox®, Aprotex®, Antagosan®, Contrycal®, Traskolan®, and others for preparing the APC or the parenteral drug of the present invention.

Another aspect of the invention is a method for preparing the PCL by dissolving APR, BCN and/or RDV in the form of a powder, a lyophilizate, or the water solution, and excipients in water or saline followed by lyophilization of the resulting mixture.

Another aspect of the invention is a method for preparing the PCL by dissolving BCN and/or RDV in the form of a powder, a lyophilizate, or the water solution, and excipients in Trasylol®, Gordox®, Aprotex®, Antagosan®, Contrycal®, or Traskolan®, and others followed by lyophilization of the resulting mixture.

The APC can be obtained, including immediately before use, by sequential dissolution in physiological solution of the crystalline APR or its lyophilizate, the crystalline BCN and/or RDV or their lyophilizate and, if necessary, excipients.

The APC can be obtained, including immediately before use, by dissolving the crystalline BCN and/or RDV or their lyophilizate in an aqueous solution of APR or in known drugs that are aqueous solutions of APR, for example, Trasylol®, Gordox®, Aprotex®, Antagosan®, Contrycal®, or Traskolan®, and others, and, if necessary, bringing the resulting compositions to the required concentration of active ingredients with saline.

The new APC can be obtained, including immediately before use, by sequential dissolution in saline of crystalline APR or its lyophilizate, for example, Contrykal®, the crystalline BCN and/or RDV or their lyophilizate and, if necessary, excipients.

During intraperitoneal treating with LPC the transgenic mice (B6.Cg-Tg(K18-ACE2)2Prlmn/HEMI Hemizygous for Tg(K18-CE2)2Prlmn from Jackson Immunoresearch, West Grove, PA, USA; females, age-6-8 weeks, weighing 19-24 g) infected with mouse-adapted SARS-CoV-2 (“Dubrovka” strain, identification number GenBank: MW161041.1) a statistically significant reduction in virus titer by 1.74 orders of magnitude was obtained in the lungs of infected animals, compared with the control group of infected but untreated animals.

During intravenous treating with LPC the Syrian hamsters weighing 100-120 g (State Scientific Center for Virology and Biotechnology “Vector” of Rospotrebnadzor, Russia) infected with SARS-CoV-2 (strain hCoV-19/Australia/VIC01/2020), a statistically significant reduction in virus titer by order of magnitude was obtained in the lungs of infected animals, compared with the control group of infected but untreated animals.

Below are examples of the preparation and use of an anti-SARS-CoV-2/COVID-19 pharmaceutical composition, confirming but not limiting the invention.

### EXAMPLE 1

Preparation of the anti-SARS-CoV-2/COVID-19 pharmaceutical composition (APC-1-APC-6, PCL-4).

APC-1. RDV from Zenji Pharmaceuticals (Suzhou) Ltd, China (22.0 mg), and APR from Wanhua Biochem, China (185.19 mg, 1,000,000 KIU) with an activity of 5400 KIU/mg were dissolved in 40 ml of a 20% aqueous solution of 2-hydroxy-beta-cyclodextrin under ultrasonic stirring for 5 minutes. To the resulting solution, ˜60 ml of saline was added to a total volume of 100 ml. The resulting mixture was stirred with ultrasound for 5 minutes to yield 100 ml APC-1 containing RDV (0.224 mg/ml) and APR (10,000 KIU/ml).

APC-2. 19 ml saline was added to 1 ml APC-1 under ultrasonic stirring to yield 20 ml APC-2 containing RDV (11.2 μg/ml) and APR (500 KIU/ml).

APC-3. Saline (40 ml) was slowly added along the inner wall of a bottle containing a lyophilisate of RDV from Zenji Pharmaceuticals (Suzhou) Ltd, China (100 mg). The vial was vigorously shaken until the drug was completely dissolved. The resulting RDV solution was added with stirring to a mixture of Trasylol®, Gordox®, Aprotex®, or Traskolan (50 ml) containing 500,000 KIU APR and 10 ml of saline to yield 100 ml of APC-3 containing RDV (1.0 mg/ml) and APR (5000 KIU/ml).

APC-4 and PCL-4. RDV from Zenji Pharmaceuticals (Suzhou) Ltd, China (50.0 mg) and APR from Wanhua Biochem, China (92.6 mg, 500,000 KIU), with an activity of 5400 KIU/mg were dissolved in a 20% aqueous solution of 2-hydroxy-beta-cyclodextrin (50 ml) under ultrasonic stirring for 5 minutes to yield 50 ml of APC-4 containing RDV (1.0 mg/ml) and APR (10,000 KIU/ml). The resulting APC-4 was lyophilized to give PCL-4 containing 50 mg RDV and 500,000 KIU APR.

APC-5. 40 mg RDV and SBECD 2.4 g sulfobutylether-β-cyclodextrin (SBECD) were added with vigorous stirring, to Gordox (20 ml) containing 10000 KIU/ml of APR. Received 20 ml of APC 5 including 2.0 mg/ml RDV and 10000 KIU/ml APR.

APC-6. RDV (50 mg) and 3 g SBECD were added with vigorous stirring, to Gordox (10 ml) containing 100000 KIU/ml of APR. Received 10 ml of APC-6, including 5.0 mg/ml RDV and 10000 KIU/ml APR.

### EXAMPLE 2

Stability of APC-2.

The stability of APC-2 and its APR and RDV components was studied by UV spectroscopy on an Agilent 8453 spectrophotometer after storage under normal conditions and under stress tests. The UV spectra of APC-2 containing 11.2 μg/ml RDV and 500 KIU/ml APR as well as the UV spectra of RDV (11.2 μg/ml) in a 0.9% aqueous sodium chloride solution were obtained under identical conditions (Table 2).

UV spectroscopic data indicate the stability of APR solutions, since the optical densities of the maxima of the initial spectrum practically coincide with those under stress test conditions.

In contrast to APR, the optical densities of the maxima of the initial spectra (Conditions 1) of RDV and APC-2 differ greatly from those in the stress test. The percentage of change in optical density under Conditions 4-9 compared to optical density under Conditions 1 is Δ>2% (Table 2).

This indicates that RDV and APC-2 are limitedly stable under rapid test conditions.

The results obtained indicate that anti-RNA viral pharmaceutical compositions APC-1-APC-6 must be used immediately after preparation.

TABLE 2
Optical density at the maxima of absorption bands in the UV spectra of APC-2
and its RDV component immediately after solution preparation (1) and after
exposure for 24 hours in the light at 25° C. (2), after exposure for
24 hours in the light at 60° C. (3), after exposure for 96 hours in the
dark at 25° C. (4), after exposure for 96 hours in the light at 25°
C. (5), after exposure for 96 hours in the dark at 3-5° C. (6), after
exposure for 96 hours in the dark at 30° C. (7), after exposure for 96
hours in the dark at 40° C. (8), and after exposure for 96 hours in the
dark at 60° C. (9). Δ, % is the percentage of change in optical density
under Conditions 2-9 compared to optical density under Conditions 1.

| | Maxima in UV spectra and percentage of change in absorbance (Δ) | | | | | |
|---|---|---|---|---|---|---|
| | under Conditions 2-9 compared to absorbance under Conditions 1 | | | | | |
| Conditions | 205 nm | Δ, % | 246 nm | Δ, % | 277 nm | Δ, % |

Optical density
RDV

| | | | | | | |
|---|---|---|---|---|---|---|
| 1 | 0.32854 | | 0.61757 | | 0.10833 | |
| 2 | 0.29007 | −1.62 | 0.60780 | −1.58 | 0.08554 | −21.04 |
| 3 | 0.32519 | −0.14 | 0.62401 | 1.04 | 0.09754 | −9.97 |
| 4 | 0.34390 | 0.65 | 0.63372 | 2.62 | 0.11012 | 1.65 |
| 5 | 0.34772 | o.81 | 0.64088 | 3.78 | 0.11622 | 7.28 |
| 6 | 0.33840 | 0.42 | 0.63125 | 2.22 | 0.10840 | 0.06 |
| 7 | 0.35053 | 0.93 | 0.64075 | 3.75 | 0.11418 | 5.40 |
| 8 | 0.34527 | 0.71 | 0.63711 | 3.17 | 0.11355 | 4.82 |
| 9 | 0.33872 | 0.43 | 0.61887 | 0.21 | 0.11323 | 4.52 |

APC-2

| | | | | | | |
|---|---|---|---|---|---|---|
| 1 | 2.56763 | | 0.65448 | | 0.18262 | |
| 2 | 2.59187 | 0.94 | 0.65258 | −029 | 0.16234 | −11.10 |
| 3 | 2.53007 | −1.46 | 0.66473 | 1.57 | 0.17357 | −4.96 |
| 4 | 2.57963 | 0.47 | 0.68294 | 4.35 | 0.18744 | 2.64 |
| 5 | 2.62453 | 2.22 | 0.68834 | 5.17 | 0.19430 | 6.40 |
| 6 | 2.62427 | 2.21 | 0.67905 | 3.75 | 0.19991. | 3.99 |
| 7 | 2.58340 | 0.61 | 0.68624 | 4.85 | 0.19040 | 4.26 |
| 8 | 2.62677 | 2.30 | 0.70740 | 8.09 | 0.20723 | 13.48 |
| 9 | 2.60123 | 1.31 | 0.67709 | 3.45 | 0.19907 | 9.00 |

### EXAMPLE 3

A device for inhalation therapy and prevention of SARS-CoV-2/COVID-19.5-10 ml of APC-4 is placed into a compression nebulizer Omron NE-C300 Complete or in a portable ultrasonic Feellife Aerogo mesh nebulizer and is receive the device for inhalation therapy and prevention of of SARS-CoV-2/COVID-19.

### EXAMPLE 4

Devices for nasal spray therapy and prevention of SARS-CoV-2/COVID-19.5-10 ml of APC-4 is placed into a plastic can for nasal and is receive the device for nasal therapy and prevention of SARS-CoV-2/COVID-19.

### EXAMPLE 5

Treatment with APC-6 of transgenic mice infected with mouse-adapted SARS-CoV-2.

In the experiment, 4 groups of transgenic mice (B6.Cg-Tg(K18-ACE2)2Prlmn/HEMI Hemizygous for Tg(K18-CE2)2Prlmn from Jackson Immunoresearch, West Grove, PA, USA), females, age—6-8 weeks, weighing 19-24 g, were formed, 4 animals per group.

Group 1—control group, untreated mice: on day 1 in the morning the mice were infected with SARS-CoV-2, and then 5 ml/kg of water for injection was intragastrically administered immediately after infection and in the evening of the same day.

Group 2—treatment with Gordox®-10 000 KIU/ml of APR. Dose: 50 000 KIU/kg APR.

Groups 3—treatment with saline solution of RDV-5 mg/ml (5_{MΓ}RDV+300_{MΓ}SBECD in 1 ml saline). Dose: 25.0 mg/kg of RDV.

Groups 4—treatment with the APC-6. Dose: 50 000 KIU/kg of APR+25 mg/kg of RDV.

Treatment regimen for transgenic mice: parenteral (intraperitoneal) administration of drugs 2 times a day; day 0-1 hour before infected with mouse-adapted SARS-CoV-2 (“Dubrovka” strain, identification number GenBank: MW161041.1) and 6-8 hours after infection; days 1, 2, 3-2 times a day, for a total of 4 days (days 0, 1, 2, 3); Day 4-lung sampling from all animals to assess the virus titer in the lungs, visual assessment of the lungs and transfer of the lungs for histology; days 0-4- daily assessment of body weight and condition of mice.

On day 0, animals from all groups were infected with the SARS-CoV-2 “Dubrovka” virus (10^{3.5} TCID₅₀/ml). All mice were infected intranasally under light ether anesthesia in a volume of 30 μl for both nostrils.

Euthanasia (painless killing of the animal) was carried out by the responsible person in accordance with the existing ethical requirements, by dislocation of the cervical vertebrae with preliminary anesthesia with ether. Euthanasia was performed promptly after the end of the experiments.

On day 4 post-infection with the virus, the animals in each group were sacrificed and the lungs were removed under sterile conditions. One lung was fixed in formalin for further histology, the second lung was prepared to measure the virus titer. To do this, after washing three times in a solution of 0.01 M phosphate buffered saline (PBS), the lungs were homogenized and resuspended in 1 ml of cold sterile PBS. The suspension was cleared from cell debris by centrifugation at 2000 g for 10 min, and the supernatant was used to determine the infectious titer of the virus in cell culture and to perform PCR. The obtained samples were stored at −80° C. until the experiments were carried out.

To determine the infectious titer of the virus from the lungs of mice, Vero CCL81 cells were seeded in 96-well Costar plates with an average density of 20,000 cells per well and grown in DMEM medium in the presence of 5% fetal calf serum, 10 mM glutamine and antibiotics (penicillin 100 IU/well). ml and streptomycin 100 μg/ml) until a complete monolayer is formed (within 3 days). Before infection with the virus, the cell culture was washed twice with DMEM medium without serum. 10-fold dilutions of each lung virus sample were prepared from 10-1 to 10-7. The prepared dilutions in a volume of 200 μL were added to cell culture plates and incubated in 5% CO2 at 37° C. for 5 days until a cytopathic effect (CPE) appeared in virus control cells. Accounting for the result of the manifestation of CPP in cells was carried out using a quantitative MTT test. The virus titer was calculated using the Ramakrishnan M. A formula in the Excel program [M. A. Ramakrishnan. Determination of 50% endpoint titer using a simple formula. World J. Virol. 2016, 5, 85-86. and expressed as 1 gTCID₅₀/ml (TCID₅₀—The median tissue culture infectious dose is defined as the dilution of a virus required to infect 50% of a given cell culture.) [I. Leneva et al. Antiviral Activity of Umifenovir In Vitro against a Broad Spectrum of Coronaviruses, Including the Novel SARS-CoV-2 Virus. Viruses 2021, 13(8): 1665. Next, the average titer value for samples from mice of the same group was calculated.

The effectiveness of the drugs in a model of the transgenic mice infected with mouse-adapted SARS-CoV-2 was assessed according to decrease in the titer of the virus in the lungs of animals after 4 days.

The obtained digital data were subjected to statistical processing in the “Statistica 8.0” software. The results are shown in Table 2.

TABLE 2
The effectiveness of the drugs and control (untreated mice) on the
model the transgenic mice infected with mouse-adapted SARS-CoV-2.

| | | | ^{b} Decrease in |
|---|---|---|---|
| Group | Dose of drug | logTCID₅₀/ml | logTCID50/mL |
| 1 | Control | 8.83, 9.50, 8.17, 8.5, 8.83 (8.77) | — |
| 2 | ^{a}APR | 7.17, 6.50, 8.17, 7.17 (7.25) | 1.52 |
| 3 | 25 mg/kg RDV | 6.83, 6.50, 6.83, 5.50, 6.17 (6.37) | 2.40 |
| 4 | ^{a}APR + 25 mg/kg RDV | 6.83, 7.83, 6.83, 7.50, 6.17 (7.03) | 1.74 |

^{a}50000 KIU/kg APR.
^{b} % reduction in virus titer (logTCID₅₀/ml) compared to control (group 1).

As can be seen from Table 2, parenteral (intraperitoneal) treatment of mice infected with SARS-CoV-2 resulted in a reduction in virus titer in the lungs of infected animals compared to a control group of infected but untreated animals. Thus, with parenteral monotherapy with APR or RDV, a decrease in virus titer (logTCID₅₀/ml) by 1.52 and 2.40, respectively, was observed. At the same time, combination parenteral therapy of APR+RDV provides a decrease in virus titer by 1.74 orders of magnitude.

### EXAMPLE 6

Intravenous treatment of Syrian hamsters infected with the SARS-CoV-2 using the PPC 8 from Example 1 containing RDV and APR.

The efficacy of APC-4 of the present invention was evaluated using a model of SARS-CoV-2 infection in Syrian hamsters [R. Boudewijns et al. STAT2 signaling as double-edged sword restricting viral dissemination but driving severe pneumonia in SARS-CoV-2 infected hamsters. BioRxiv preprint; this version posted Apr. 24, 2020.

The strain SARS-CoV-2 hCoV-19/Australia/VIC01/2020 was obtained from the State Research Center of Virology and Biotechnology VECTOR (Russia). The infectious virus was isolated by sequential passage in Vero E6 cells. The titer of the viral suspension was determined by endpoint dilution on Vero E6 cells using the Reed-Muench method. The work related to the live virus was carried out under isolated laboratory conditions that meet the international BSL-330 VECTOR requirements.

Vero E6 cells from VECTOR's Collection of Cell Cultures were cultured in Minimum Essential Medium (MEM) (Gibco) supplemented with 10% fetal bovine serum (Integro), 1% L-glutamine (Gibco), and 1% Bicarbonate (Gibco). Endpoint titrations were performed with a medium containing 2% fetal bovine serum.

Wild-type Syrian hamsters at the age of 6-10 months weighing 100-120 g from State Scientific Center for Virology and Biotechnology “Vector” of Rospotrebnadzor (Russia) were kept with unlimited access to food and water. Hamsters were randomized into 4 cohorts, 8 animals in each cohort (4 males and 4 females).

Hamsters were anesthetized with zoletil-xyla and inoculated into each nostril with 50 μl anesthetic combination containing 10³TCID₅₀.

Group 1—control group, untreated hamsters. Dose: 5 ml/kg saline.

Group 2—treatment with Gordox®-10 000 KIU/ml of APR. Dose: 10000 KIU/kg APR.

Groups 3—treatment with saline solution of RDV-1 mg/ml. Dose: 5 mg/kg of RDV.

Groups 4—treatment with the APC-4 (RDV-1 mg/ml +10000 KIU/ml of APR). Dose: 50 000 KIU/kg of APR +5 mg/kg of RDV.

The drugs were injected under light isoflurane anesthesia intravenously, 2 times a day for 4 days, starting the first injection one hour before infection, 6 hours after infection, then for 3 days after 12 hours.

Hamsters were checked daily for appearance, behavior and weight. On the 4th day after infection, the hamsters were euthanized by intravenous injection of 500 μl doletal (200 mg/ml sodium pentobarbital, Vétoquinol SA). Hamster lung tissues were harvested after sacrifice and homogenized using a Precellys homogenizer in a 350 μl RNeasy lysis buffer (RNeasy Mini kit, Qiagen) and centrifuged (10,000 rpm, 5 min) to remove cell debris. RNA was extracted according to the manufacturer's instructions. Real-time PCR was performed on the LightCycler96 platform (Roche) using the iTaq Universal Probes One-Step RT-qPCR kit (BioRad) [R. Boudewijns et al. STAT2 signaling as double-edged sword restricting viral dissemination but driving severe pneumonia in SARS-CoV-2 infected hamsters. BioRxiv preprint; this version posted Apr. 24, 2020.

For histological analysis, lung tissue was fixed in 4% formaldehyde, embedded in paraffin, and stained with hematoxylin-eosin. Damage was assessed on a scale from 1 to 3: stagnation, intraalveolar bleeding, apoptotic bodies in the bronchial epithelium, necrotic bronchiolitis, perivascular edema, bronchopneumonia, perivascular inflammation, peribronchial inflammation, and vascular inflammation.

Statistical analysis was performed using the GraphPed Prism software from GraphPed Software, Inc. Statistical significance was determined using the Mann-Whitney nonparametric U-test. The values of P≤0.05 were considered significant.

An analysis of the results obtained showed that APC-4 comprising RDV and APR demonstrated a high anti-SARS-CoV-2/COVID-19 efficacy. Thus, in comparison with the control group, after intravenous treatment of Syrian hamsters infected with the SARS-CoV-2 of drug PPC 1, the titer of SARS CoV-2 in the tissues of the lungs decreased by more than an order of magnitude compared to the control group.

1-10. (canceled)

11. A pharmaceutical composition for the treatment of Coronavirus Disease of 2019 (COVID-19) infection comprising a therapeutically effective amount of aprotinin and remdesivir, or pharmaceutically acceptable salts thereof, and an excipient.

12-13. (canceled)

14. A pharmaceutical kit comprising a therapeutically effective amount of aprotinin and remdesivir, or pharmaceutically acceptable salts thereof, wherein the compounds are in a dissolved or lyophilized form.

15. The pharmaceutical kit of claim 14, wherein the kit includes an aprotinin in the dissolved form.

16-17. (Canceled)

18. A method for treating Coronavirus Disease of 2019 (COVID-19) comprising administering parenterally to a mammal a therapeutically effective amount of aprotinin and remdesivir, or pharmaceutically acceptable salts thereof.

19. (canceled)

20. The method of claim 18, wherein the aprotinin comprises a dissolved form.

21. (canceled)

22. The method of claim 18, comprising administering a therapeutically effective amount of aprotinin and remdesivir, or pharmaceutically acceptable salts thereof, via inhalation or intravenous administration.

23. The method of claim 18, comprising administering a therapeutically effective amount of aprotinin and remdesivir, or pharmaceutically acceptable salts thereof, via nasal administration.

24-26. (canceled)
